(19) United States
(12) Patent Application Publication
(10) Pub. No.: US 2020/0315954 A1
(43) Pub. Date: Oct. 8, 2020

(54) Topical Skin Compositions and Devices and Methods for Using Same

(71) Applicant: O. L. Products, Inc., Oldsmar, FL (US)

(72) Inventors: Brian May, Palm Harbor, FL (US); Anthony Gullo, Odessa, FL (US)

(73) Assignee: O. L. Products, Inc., Oldsmar, FL (US)

(21) Appl. No.: 16/373,198

(22) Filed: Apr. 2, 2019

(51) Int. Cl. A61K 9/00; A61Q 17/04; A61K 31/047; A61K 8/06; A61K 9/107; A61K 9/06; A61K 9/08; A61K 36/185; A61K 8/27; A61K 8/29; A61M 35/00

(52) U.S. Cl. CPC A61K 9/0014; A61Q 17/04; A61K 31/047; A61K 8/06; A61K 9/107; A61K 9/06; A61M 2210/04; A61K 36/185; A61K 8/27; A61K 8/29; A61M 35/003; A61K 2800/87; A61K 2800/882; A61K 9/08

(57) ABSTRACT

The present disclosure provides topical skin formulations including sunscreen compositions and cannabidiol compositions. The present disclosure further provides topical skin formulations including both sunscreen agents and cannabidiol. The sunscreen agents are included in an amount from about 0.01 to 20% by weight. Cannabidiol is included in an amount from about 0.01 to 10% by weight. A method for applying multiple topical skin formulations includes separately storing a sunscreen composition and a cannabidiol composition in a multi-chambered device. The multi-chambered device may be configured to dispense each composition independently of the other or simultaneously in adjustable ratios.

### FIELD

The present disclosure relates to a topical skin composition and, more particularly, to devices and methods for using a sunscreen composition and a cannabidiol composition.

### BACKGROUND

It has long been known that prolonged exposure to ultraviolet (UV) radiation from the sun damages skin. Depending on geographic location and occupation, some individuals must spend long hours in the sun. Many individuals also endure prolonged exposure to sunlight for recreational or leisure activities.

Sunscreen compositions have been developed to protect the skin from the sun's damaging rays. Sunscreen compositions include one or more sunscreen agents in a dermatologically acceptable vehicle. Sunscreen agents work by absorbing or reflecting UV rays. Sunscreen products are topically applied on skin that will be exposed to the sun and the sunscreen agents therein provide protection from the UV rays. In addition to sunscreen, various other products with a myriad of intended benefits are also topically applied on skin. Some intended benefits of these products include treating pain, anxiety, nausea, epilepsy, and many other ailments.

In recent years, cannabidiol has been gaining popularity for its many health benefits. Cannabidiol (CBD) is one of the many chemical compounds found in the cannabis plant. CBD is a non-psychoactive cannabinoid that has shown promising results in treating a multitude of ailments. For example, CBD has been used to treat pain, anxiety, depression, nausea, seizures, and sleep-related disorders. Similar to sunscreen, one common way to utilize CBD is through topical application of oils, creams, or lotions.

One object of the present disclosure is to provide a topical skin composition that provides both UV protection and the health benefits associated with CBD.

Another object of the present disclosure is to provide a device and method to store and apply topical skin compositions that include UV protection and CBD.

### SUMMARY

In one aspect of the disclosure, a topical skin composition is provided that contains a sunscreen agent, cannabidiol, and a dermatologically acceptable vehicle.

In some embodiments, the dermatologically acceptable vehicle is an emulsion, a cream, a lotion, a serum, or a solution.

In some embodiments, the topical skin composition also includes Cannabis Sativa Seed Oil and/or caffeine.

In some embodiments, the topical skin composition includes about 0.01% to 10% by weight of cannabidiol.

In some embodiments, the topical skin composition includes about 0.01% to 20% by weight of a sunscreen agent.

In one embodiment, the topical skin composition the sunscreen agent is either titanium dioxide, zinc oxide, or a combination thereof.

In one embodiment, the topical skin composition includes about 0.01% to 20% by weight of titanium dioxide and about 0.01% to 15% by weight of zinc oxide.

In another aspect of the disclosure, a method of using more than one topical skin composition is provided. First, a multi-chambered device configured to store a first composition in a first chamber and a second composition in a second chamber is provided. The first composition is dispensed from a first chamber dispenser; and the second composition is dispensed from a second chamber dispenser. The first composition and the second composition may be dispensed individually or simultaneously. Once the first composition and the second composition have been dispensed onto an area of skin, the first composition and the second composition are applied to the area of skin.

In some embodiments with simultaneous dispensing of the two compositions, the multi-chambered device includes an adjustable head that can change the ratio of first composition to second composition dispensed in a single dispensing action.

In yet another aspect of the disclosure, a method of storing and applying topical skin compositions including sunscreen agents and cannabidiol is provided. First, a multi-chambered device is provided. A first chamber connected to a first dispenser is filled with a sunscreen composition; and a second chamber connected to a second dispenser is filled with a cannabidiol composition. Then, the sunscreen composition and the cannabidiol composition are dispensed onto an area of skin to be rubbed into the skin thereby providing health benefits of cannabidiol and protection from UV radiation.

### BRIEF DESCRIPTION OF THE DRAWINGS

FIG. 1 illustrates a device to store and apply topical skin compositions of sunscreen and CBD; and

FIG. 2 illustrates a device to store separate topical skin compositions that are dispensed with a single dispensing action.

### DETAILED DESCRIPTION

In accordance with the present disclosure, a topical skin composition comprising CBD, a sunscreen agent, and a dermatologically suitable vehicle is provided for treating health ailments while providing protection from UV light. Included in the present disclosure are devices to store and apply a topical skin composition such as an ointment, lotion, cream, serum, salve, balm, tincture, or gel formulation.

In one embodiment, the topical skin composition contains CBD, a sunscreen agent, and a dermatologically acceptable vehicle. The compositions of the present invention can be formulated into all types of vehicles. Non-limiting examples of suitable vehicles include emulsions (e.g., oil-in-water, water-in-oil, silicone-in-water, water-in-silicone, water-in-oil-in-water, oil-in-water, oil-in-water-in-oil, oil-in-water-in-silicone, etc.), creams, lotions, solutions (both aqueous and hydro-alcoholic), anhydrous bases (such as lipsticks and powders), gels, ointments, pastes, milks, salves, balms, tinctures, liquids, aerosols, solid forms, or jellies. Variations and other appropriate vehicles will be apparent to the skilled artisan and are appropriate for use in the present invention. In certain aspects, the concentrations and combinations of the ingredients can be selected in such a way that the combinations are chemically compatible and do not form complexes which precipitate from the finished product.

In general, the topical skin composition suitable for use in accordance with the present disclosure may be applied in any dermatologically acceptable vehicle such as a serum, cream, ointment, gel, lotion, or pad applied formulation, which may or may not be emulsified and may contain ingredients to improve, modify, or stabilize the formulation physically or cosmetically. Other suitable formulations will be apparent to those skilled in the art.

In some embodiments, the topical skin composition can include a mixture of sunscreen agents and inactive ingredients. Exemplary sunscreen agents include, but are not limited to, zinc oxide, titanium dioxide, benzophenones, avobenzone, cinnamates, salicylates, and octocrylene. Exemplary inactive ingredients include, but are not limited to, water, glycerin, Helianthus Annuus seed oil, Vitis Vinifera seed oil, alumina, silica, glyceryl stearate, tocopherol, xanthan gum, tetrasodium EDTA, phenoxyethanol, and ethylhexylglycerin.

In some embodiments, the sunscreen agents are titanium dioxide and zinc oxide.

In one exemplary embodiment, the topical skin composition includes CBD, titanium dioxide, zinc oxide, water, glycerin, Helianthus Annuus seed oil, Vitis Vinifera seed oil, alumina, silica, glyceryl stearate, tocopherol, xanthan gum, tetrasodium EDTA, phenoxyethanol, and ethylhexylglycerin. In one exemplary embodiment, the topical skin composition includes from about 0.01 to 10% by weight CBD, from about 0.01 to 20% by weight of titanium dioxide, and from about 0.01 to 15% by weight of zinc oxide.

In another aspect of this disclosure, a device is provided to store, transport, and apply separate compositions of sunscreen and CBD to achieve protection from UV rays and health benefits of CBD. An exemplary CBD composition is comprised of Cannabis Sativa seed oil, cannabidiol, caffeine, and a dermatologically acceptable vehicle.

Table 1 illustrates the ingredients and the amount of each ingredient by weight percent of another exemplary CBD composition.

TABLE 1
Exemplary CBD Composition

| | Amount |
|---|---|
| Ingredient | (percent by weight) |
| Water | 68.02% |
| Cannabis Sativa Seed Oil | 11.00% |
| Stearic Acid | 4.00% |
| Dimethicone | 3.33% |
| Glycerin | 2.44% |
| Propanediol | 2.44% |
| PEG-100 Stearate | 1.80% |
| Sodium Acrylate/Sodium Acryloyldimethyl | 1.56% |
| Taurate Copolymer | |
| Theobroma Cacao Seed Butter | 1.00% |
| Butyrospermum Parkii Butter | 1.00% |
| Glyceryl Stearate | 0.80% |
| Cannabidiol | 0.50% |
| Caffeine | 0.22% |
| Isopropyl Palmitate | 0.89% |
| Phenoxyethanol | 0.90% |
| Ethylhexylhlycerin | 0.10% |

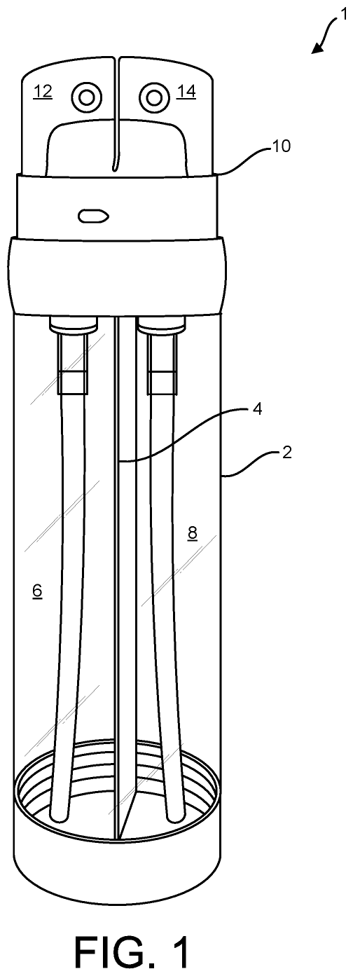

Referring to FIG. 1, an exemplary device is shown that separately stores a sunscreen composition and a CBD composition for independent application of each composition. The device 1 has a cavity 2 divided by a wall 4 into two separate chambers 6/8. The first chamber 6 contains one of either the sunscreen composition or the CBD composition; the second chamber 8 contains the other composition. A lid 10 affixes to the top of the chamber 2 and is configured to dispense the compositions in the chambers 6/8. The lid 10 is configured to separately dispense the sunscreen composition and CBD composition by pumping, spraying, pouring, or any other suitable method.

By way of example, the lid 10 in FIG. 1 shows a lid 10 with dual pumps 12/14. First pump 12 is configured to dispense the composition from first chamber 6. And second pump 14 is configured to dispense the composition from second chamber 8.

A method of applying the sunscreen composition and the CBD composition includes dispensing a desired amount of sunscreen composition from the corresponding pump 12/14 onto the skin or hand. Then, a desired amount of CBD composition is dispensed from the other pump 12/14. The sunscreen and CBD compositions are then applied simultaneously to the skin providing both UV protection and the health benefits of CBD.

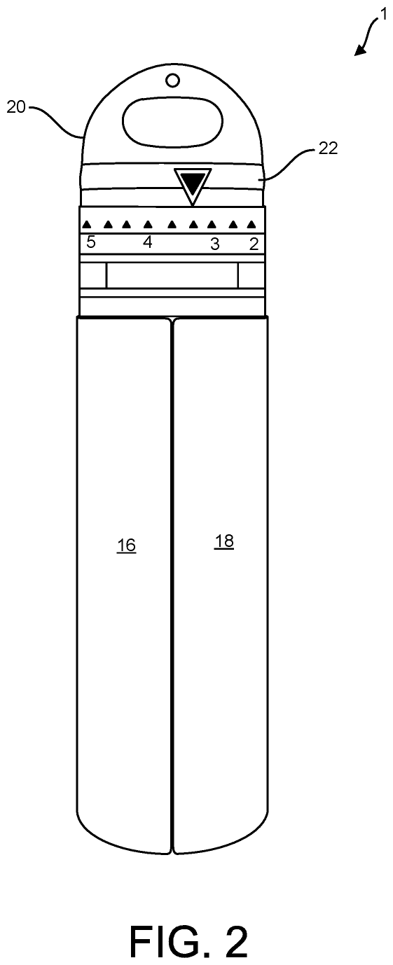

In another embodiment, a sunscreen composition and a CBD composition can be stored separately but applied using a single dispensing action. Referring to FIG. 2, an exemplary device is shown that is configured to dispense the sunscreen composition and the CBD composition in a single dispensing action. The device 1 is comprised of a first chamber 16 and a second chamber 18. The chambers 16/18 are configured to fit together into a head 20. The first chamber 16 contains one composition and the second chamber 18 contains another composition. For example, the first chamber 16 can contain a sunscreen composition while the second chamber contains a CBD composition. When the chambers 16/18 are affixed to the head 20, the head 20 is configured to dispense the composition of the first chamber 16 and the composition of the second chamber 18 in a single dispensing action (e.g. a depression of a pump portion of the head 20).

In one embodiment, the head 20 can be configured to dispense an equal amount from each chamber 16/18. In another embodiment, the head 20 can be configured to dispense a different amount of each composition from the respective chamber 16/18. In yet another embodiment, the head 20 is adjustable such that a user can change the ratio of how much of each composition is dispensed from a respective chamber 16/18. By way of example, the head 20 may be rotated clockwise or counterclockwise to change the amount of composition that will be drawn out of each chamber during the dispensing action.

A method of applying a sunscreen composition and a CBD composition using the adjustable ratio device includes rotating the head 20 clockwise or counterclockwise to the desired ratio 22 of CBD composition to sunscreen composition. Then, dispense the sunscreen composition and the CBD composition by depressing the head 20. Finally, the compositions are applied on the skin providing both UV protection and the health benefits of CBD.

Many different embodiments have been disclosed herein, in connection with the above description and the drawings. It will be understood that it would be unduly repetitious and obfuscating to literally describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

It will be appreciated by persons skilled in the art that the embodiments described herein are not limited to what has been particularly shown and described herein above. In addition, unless mention was made above to the contrary, it should be noted that all of the accompanying drawings are not to scale. A variety of modifications and variations are possible in light of the above teachings.

What is claimed is:

1. A topical skin composition comprising a sunscreen agent, cannabidiol, and a dermatologically acceptable vehicle.

2. The topical skin composition of claim 1, wherein the dermatologically acceptable vehicle is selected from the group consisting of an emulsion, a cream, a lotion, a serum, and a solution.

3. The topical skin composition of claim 1, further comprising Cannabis Sativa Seed Oil.

4. The topical skin composition of claim 1, wherein the topical skin composition includes from about 0.01% to about 10% by weight of cannabidiol.

5. The topical skin composition of claim 1, wherein the topical skin composition includes from about 0.01% to about 20% by weight of the sunscreen agent.

6. The topical skin composition of claim 1, wherein the sunscreen agent is selected from the group consisting of titanium dioxide and zinc oxide.

7. The topical skin composition of claim 1, wherein the sunscreen agent includes zinc oxide and titanium dioxide.

8. The topical skin composition of claim 7, wherein the sunscreen agent includes from about 0.01% to about 20% by weight of titanium dioxide and from about 0.01% to 15% by weight of zinc oxide.

9. A method of using more than one topical skin composition, the method comprising the steps of:
providing a multi-chambered device configured to store a first composition in a first chamber and a second composition in a second chamber;
dispensing the first composition from a first chamber dispenser;
dispensing the second composition from a second chamber dispenser; and
applying the first composition and the second composition to an area of skin.

10. The method of claim 9, wherein the multi-chambered device is configured to dispense the first composition and the second composition simultaneously.

11. The method of claim 10, wherein the multi-chambered device includes an adjustable head, the adjustable head configured to change the ratio of first composition to second composition when the first composition and second composition are dispensed simultaneously.

12. The method of claim 9, wherein the first composition is a sunscreen formulation.

13. The method of claim 12, wherein the second composition includes Cannabis Sativa seed oil.

14. The method of claim 13, wherein the second composition is a cannabidiol formulation.

15. The method of claim 13, wherein the sunscreen formulation includes titanium dioxide and zinc oxide.

16. The topical skin composition of claim 13, wherein the sunscreen formulation includes from about 0.01% to about 20% by weight of titanium dioxide and from about 0.01% to 15% by weight of zinc oxide.

17. A method of storing and applying topical skin compositions including sunscreen agents and cannabidiol, the method comprising the steps of:
providing a multi-chambered device having a first chamber connected to a first dispenser and a second chamber connected to a second dispenser;
filling the first chamber with a sunscreen composition;
filling the second chamber with a cannabidiol composition;
dispensing the sunscreen composition onto an area of skin;
dispensing the cannabidiol composition onto an area of skin; and
applying the sunscreen composition and the cannabidiol composition into the area of skin.

18. The method of claim 17, wherein the sunscreen composition includes titanium dioxide and zinc oxide.

19. The topical skin composition of claim 17, wherein the sunscreen composition includes from about 0.01% to about 20% by weight of titanium dioxide and from about 0.01% to 15% by weight of zinc oxide.

20. The method of claim 17, wherein the cannabidiol composition includes Cannabis Sativa seed oil and caffeine.
